(19) United States
(12) Patent Application Publication
(10) Pub. No.: US 2011/0104081 A1
(43) Pub. Date: May 5, 2011

(54) Oral Compositions for Treatment of Dry Mouth

(76) Inventors: Douglas Craig Scott, Loveland, OH (US); David Salloum Salloum, West Chester, OH (US); Ann Gilligan Snider, Loveland, OH (US); Christine Lula Coupp, Miamisburg, OH (US)

(21) Appl. No.: 12/914,669

(22) Filed: Oct. 28, 2010

Related U.S. Application Data
(60) Provisional application No. 61/257,677, filed on Nov. 3, 2009.

(51) Int. Cl. A61K 8/22; A61K 8/81; A61Q 11/00

(52) U.S. Cl. 424/53; 424/49

(57) ABSTRACT

An oral care composition for the treatment of dry mouth comprising a polyethylene oxide with a molecular weight from about 200,000 to about 7,000,000, an anti-bacterial agent, and a sensate.

### CROSS REFERENCE TO RELATED APPLICATION

This application claims the benefit of provisional application Ser. No. 61/257,677, filed on Nov. 3, 2009, which is incorporated by reference in its entirety herein.

### FIELD OF THE INVENTION

The present invention relates to oral compositions, such as dentifrices and rinses, and methods of use, that provide treatment for dry mouth, the oral compositions comprising polyethylene oxide with a molecular weight from about 200,000 to about 7,000,000, an antibacterial agent, and a sensate.

### BACKGROUND OF THE INVENTION

Dry mouth, also known as xerostomia, is a common condition indicated by a feeling that there is not enough saliva in the mouth. Most everyone experiences dry mouth in the morning. Many people complain of dry mouth also during the day. Consumers complaining of daily dry mouth range from 32% in the US to 72% in China. The prevalence of dry mouth increases with age, particularly for those over 35, due to a natural decrease in salivary flow. For young adults, about 20 years old, 15-20% complain of dry mouth; for those 60-80 years old, roughly 30-40% suffer from dry mouth. Medications taken can also cause or increase dry mouth. Over 400 types of medicine have the side effect of dry mouth, including medicines for allergies, asthma, blood pressure, pain killers, anti-depressants, diuretics, and mood-altering drugs. Moreover, drinking alcohol or caffeine or smoking also increase the incidence of dry mouth.

The consequences of dry mouth are generally unpleasant for people and pose quality of life issues. Dry mouth increases the risk of caries and infection, particularly of yeast infections. Dry mouth also contributes to a higher risk for periodontal disease. In addition, dry mouth can cause bad breath or a bad taste in the mouth.

Therefore, there is a need for oral care compositions that can treat and lessen the effects of dry mouth. The present invention relates to oral care compositions that treat dry mouth, both the perception of dry mouth and the functional consequences. The present invention uses a multi-prong approach, with compositions comprising high molecular weight polyethylene oxides that improve mouth moisturization and lubricity, high bioavailable antibacterials and anti-inflammatory agents to boost hydration in the mouth, and sensates, such as saliva stimulants, flavors, and TRPV1 activators to reduce dryness perception. The present invention comprehensively treats dry mouth by both the functional and aesthetic benefits of the combination of high molecular weight PEO's, antibacterial agents, and sensates.

### SUMMARY OF THE INVENTION

The present invention relates to an oral care composition for the treatment of dry mouth comprising polyethylene oxide with a molecular weight from about 200,000 to about 7,000,000, an antibacterial agent, and a sensate.

### DETAILED DESCRIPTION OF THE INVENTION

While the specification concludes with claims that particularly point out and distinctly claim the invention, it is believed the present invention will be better understood from the following description.

### DEFINITIONS

The term “orally acceptable carrier” as used herein means a suitable vehicle, which can be used to apply the present compositions to the oral cavity in a safe and effective manner. Such vehicle may include materials such as fluoride ion sources, antibacterial agents, anticalculus agents, buffers, other abrasive materials, peroxide sources, alkali metal bicarbonate salts, thickening materials, humectants, water, surfactants, titanium dioxide, flavor system, sweetening agents, cooling agents, xylitol, coloring agents, and mixtures thereof.

The term “comprising” as used herein means that other steps and other ingredients which do not affect the end result can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “effective amount” as used herein means an amount of a compound or composition sufficient to significantly induce a positive benefit, preferably an oral health benefit, but low enough to avoid serious side effects, i.e., to provide a reasonable benefit to risk ratio, within the sound judgment of a skilled artisan.

The term “oral composition” as used herein means a product that in the ordinary course of usage is not intentionally swallowed for purposes of systemic administration of particular therapeutic agents, but is rather retained in the oral cavity for a time sufficient to contact substantially all of the dental surfaces and/or oral tissues for purposes of oral activity. The oral composition of the present invention may be in various forms including toothpaste, dentifrice, rinses, gels, edible films, candy, confectionary gums, lozenges, sprays, tooth powders, tablets, subgingival gel, foam, mouse, or denture product. The oral composition may also be incorporated onto strips or films for direct application or attachment to oral surfaces.

The term “dentifrice” as used herein means paste, gel, powder, tablets, or liquid formulations, unless otherwise specified, that are used to clean the surfaces of the oral cavity.

The term “teeth” as used herein refers to natural teeth as well as artificial teeth or dental prosthesis.

The term “polymer” as used herein shall include materials whether made by polymerization of one type of monomer or made by two (i.e., copolymers) or more types of monomers.

The term “water soluble” as used herein means that the material is soluble in water in the present composition. In general, the material should be soluble at 25° C. at a concentration of 0.1% by weight of the water solvent, preferably at 1%, more preferably at 5%, more preferably at 15%.

The term “phase” as used herein means a mechanically separate, homogeneous part of a heterogeneous system.

The phrase “bioavailable” and its variants means the focal chemical entity, such as an element, an ion, or a molecule, is available for therapeutic efficacy. For example, bioavailable stannous results when stannous ions dissociate from the stannous ion source and then proceed to chemically interact with the tooth surface and/or gum tissue. Bioavailable stannous includes stannous cations having a valence of +2. Stannous cations that have been oxidized to the +4 valence or are over-stabilized by a chelating agent have lower bioavailability.

The term “sensate” as used herein means refers to a material in which its predominant effect in the oral cavity is to impart a sensation, for example, a taste, moisturization, warming, cooling, and/or tingling sensation. A sensate may be, but is not limited to, a flavor, a sweetener, a coolant, a saliva stimulant, or a TRPV1 activator.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

### High Molecular Weight PEO's

In some embodiments, the present invention comprises a high molecular weight polyethylene oxide. Polyethylene oxide (PEO) may alternatively be called polyoxyethylene or polyethylene glycol. The use of polyethylene oxides in oral care compositions is known in the art, but typically, they have been of relatively low molecular weight, generally from about 200 to about 700.

The present inventors have discovered that the use of high molecular weight PEO's, with molecular weights from about 200,000 to about 7,000,000, provide several benefits to lessen dry mouth. First, the high molecular weight PEO's lubricate the mouth. This lubrication, meaning the lack of friction between elements in contact, provides the opposite effect of dryness. In addition, the high molecular weight PEO's provide actual mouth moisturization by retaining water. Other materials that have been used to treat dry mouth and/or to lubricate the mouth, such as carboxymethylcellulose, for example, do not retain water as well as high molecular weight PEO's. Table 1 shows that polyox 301 (a PEO of approximate molecular weight of 4,000,000) has better moisture retention than carboxymethylcellulose by demonstrating that polyox 301 has less water loss on model substrates (cotton).

TABLE 1
Table 1: Water loss, in %, from cotton substrates treated
with polymer solutions containing 0.1% polymer by weight.

| | Wet | 1 hr | 2 hrs | 3 hrs | 4 hrs | 5 hrs | 6 hrs | 7 hrs | 8 hrs | 24 hrs |
|---|---|---|---|---|---|---|---|---|---|---|
| PEO | 0.0 | 1.79 | 4.36 | 7.52 | 10.11 | 13.45 | 16.76 | 20.47 | 24.66 | 57.78 |
| CMC | 0.0 | 1.90 | 4.81 | 7.89 | 10.65 | 14.17 | 17.65 | 21.83 | 26.82 | 64.11 |
| Control | 0.0 | 2.43 | 5.05 | 8.21 | 11.04 | 14.38 | 17.69 | 21.53 | 26.20 | 62.23 |

The control was water treated cotton substrates. Samples in this test were monitored wet and did not go through drying process

Furthermore, when high molecular weight PEO's are used in combination with polyols (for example, glycerin, erythritol, xylitol, sorbitol, mannitol), a synergistic effect of better moisture retention is achieved, better than either PEO's or polyols used alone, or than simply an additive effect. Table 2 demonstrates this, by showing that cotton samples rinsed with water (“with rinse”) retain the most water when treated with a combination of both PEO and glycerin.

TABLE 2
Table 2: Water gain (mg) for cotton
swatches with different treatments.

| | | 40% | 0.1% | 40% glycerin + | 20% glycerin + |
|---|---|---|---|---|---|
| | Control | glycerin | PEG | 0.1% PEG | 0.05% PEG |
| No | 25.4 | 393.3 | 15.1 | 404 | 257.1 |
| Rinse | | | | | |
| With | 23.6 | 126.9 | 15.2 | 216.0 | 107.6 |
| Rinse | | | | | |

Table shows synergetic effect of PEO + glycerin on moisture uptake with retention benefit. No-rinse samples were treated, dried then incubated to monitor moisture uptake. With rinse samples were treated, rinsed with water, dried then incubated to monitor moisture uptake, Control represents swatches treated with water only.

One reason the PEO's are able to deliver superior moisture retention is because the PEO's of the present invention are retained in the soft tissue of the mouth and not easily washed away. For example, polyols are retained in the mouth and are perceived to moisturize for less than five minutes before being washed away. In contrast, PEO's are retained in the mouth for up to three hours. Therefore, consumer perception of the PEO's moisturization benefit lasts significantly longer than with polyols.

The PEO's of the present invention are high molecular weight PEO's because the higher the molecular weight, the better the PEO delivers moisture retention. The higher the molecular weight, the better the adhesion profile, meaning the PEO is retained in the mouth longer and less likely to wash away. To formulate in various oral care compositions, the preferred molecular weight is from about 900,000 to about 6,000,000, and more preferred is from about 3,000,000 to about 5,000,000. While the higher the molecular weight is, the better the adhesion is, at too high a molecular weight, the PEO is very sticky and slimy and may cause an unpleasant consumer perception, particularly if formulated in a dentifrice, for example.

The high molecular weight polyethylene oxide may be present in an amount from about 0.001% to about 5.0%, by weight of the composition. For dentifrice formulations of the present invention, the preferred amount present is from about 0.05% to about 0.5%. In some dentifrice embodiments, the amount present is from about 0.1% to 0.2%. In some embodiments, a solid dentifrice may have from about 0.5% to 1.0%, by weight of the composition, of a high molecular weight PEO. For rinse, the preferred amount present is from about 0.003% to about 0.05%, in some embodiments from about 0.005% to about 0.025%.

A further benefit of the high molecular weight PEO's is that when formulated into oral care compositions with antibacterial agents, for example such as stannous, zinc, or CPC, the PEO's do not interfere with the bioavailability of the antibacterial agents. This is very important, because part of effectively treating and lessening the effects of dry mouth is to free the mouth of harmful bacteria that can cause infection and/or caries. The low flow of saliva not only causes the unpleasant perception of dryness, but also contributes to the real problem of allowing bacteria to metabolize, leading to infections and/or caries. The high molecular weight PEO's, due to their hydrophilic and non-ionic nature, do not reduce or destroy the bioavailability of the antibacterial agents. So while high molecular PEO's offer unique lubricating and mouth moisturization benefits, a composition that offers both high molecular weight PEO's along with bioavailable antibacterial agents provides a stronger and more comprehensive treatment of the dry mouth condition.

For example, as can be seen in table 3, when an antibacterial agent such as cetylpyridinium chloride (CPC) is formulated into a rinse with a high molecular weight PEO and compared with a commercial rinse advertised to treat dry mouth (formulated with carboxymethylcellulose), the high molecular weight PEO formulations have higher bioavailability of the CPC.

TABLE 3

| | DRA |
|---|---|
| Rinses containing 500 ppm CPC | (Bioavailable CPC) |
| Formula I (0.1 PEO, 0.05 poloxamer, 18% | 89.9% |
| glycerin) | |
| Formula II (Crest Pro-Health Rinse + PEO mw 4M) | 91.6% |
| Commercial rinse containing CMC and other | 20% |
| ionic polymers | |

A further benefit of high molecular weight PEO's is that they improve negative aesthetics (astringency, bitterness, metallic taste, medicinal effect) that can occur from other common formulation components, particularly antibacterial agents such as stannous, zinc, or CPC. Even small amounts of high molecular weight PEO's can mask or reduce astringency. For example, consumer testing for rinse formulations shows that just 0.005% of polyox 301 can lessen consumer perception of bitterness and metallic and medicinal taste. And of course these negative aesthetics, particularly astringency or bitterness, contribute to the perception of dryness in the mouth. Therefore, reduction of these negative aesthetics is important for a composition that treats dry mouth, such as the present invention.

High molecular weight PEO's may be used in combination with other materials to increase or improve the PEO's performance. For example, poloxamers may be used in combination with the PEO's. Suitable poloxamers include those of the form (PEO-PPO-PEO). Examples of poloxamers that may be used include, but are not limited to, the following:

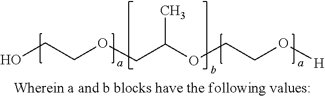

| Pluronic ® | Poloxamer | a | b |
|---|---|---|---|
| L44NF | 124 | 12 | 20 |
| F68NF | 188 | 80 | 27 |
| F87NF | 237 | 64 | 37 |
| F108NF | 338 | 141 | 44 |
| F127NF | 407 | 101 | 56 |

As mentioned above, polyols may also be used in combination with the PEO's to help maximize moisture retention. Polyols that may be used include, but are not limited to, glycerin, erythritol, xylitol, sorbitol, maltitol, isomalt, lactitol, diglycerin, hydrogenated starch hydrolysate (HSH), and mixtures thereof.

### Sensates

Because dry mouth is a condition defined by the sensation that there is not enough saliva in the mouth, the present invention may further comprise sensates that prevent or lessen the dryness and/or the dryness perception.

For example, saliva stimulants, or sialagogues, such as pellitorin, are known to be helpful to ameliorate the condition of dry mouth. Saliva stimulating agents are further disclosed in U.S. Pat. No. 4,820,506. Saliva stimulating agents suitable for use in the present invention further include, but are not limited to, fruit acids or an acid component such as phosphoric acid, adipic acid, succinic acid, citric acid, malic acid, tartaric acid, fumaric acid, lactic acid, acetic acid, cinnamic acid and mixtures thereof. Additional food acids may be found in the Source Book of Flavors, AVI Publishing Company Inc. (1981), incorporated here by reference. Other spicy substances and/or substances stimulating salivation in the mouth and/or substances that produce a sensation of heat and/or a tingling sensation on the skin or on the mucous membranes, and which may be a constituent of the preparations according to the invention, are for example: capsaicin, dihydrocapsaicin, gingerols, paradols, shogaols, piperin, carboxylic acid N-vanillylamides, in particular nonanoic acid N-vanillylamide, pellitorin or spilanthol, 2-nonenoic acid amides, in particular 2-nonenoic acid N-isobutylamide, 2-nonenoic acid N-4-hydroxy-3-methoxyphenylamide, alkyl ethers of 4-hydroxy-3-methoxybenzyl alcohol, in particular 4-hydroxy-3-methoxybenzyl-n-butyl ether, alkyl ethers of 4-acyloxy-3-methoxybenzyl alcohol, in particular 4-acetyloxy-3-methoxybenzyl-n-butyl ether and 4-acetyloxy-3-methoxybenzyl-n-hexyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylenedioxybenzyl alcohol, (4-hydroxy-3-methoxyphenyl)acetic acid amides, in particular (4-hydroxy-3-methoxyphenyl)acetic acid N-n-octylamide, vanillomandelic acid alkylamides, ferulic acid phenethylamides, nicotinaldehyde, methyl nicotinate, propyl nicotinate, 2-butoxyethyl nicotinate, benzyl nicotinate, 1-acetoxychavicol, polygodial and isodrimeninol, and also preferred are cis- and/or trans-pellitorine according to WO 2004/000787 and WO 2004/043906, alkenecarboxylic acid N-alkylamides according to WO 2005/044778, mandelic acid alkylamides according to WO 03/106404 or alkyloxyalkanoic acid amides according to WO 2006/003210.

Further non-limiting examples of salivating agents can be found in WO2009/066262A1. Examples include formula (I): (I) wherein Ri represents C1-C2 n-alkyl; R2 is 2-methyl-1-propyl and R3 is hydrogen, or R2 and R3 taken together is a moiety having the formula —(CH2)n- wherein n is 4 or 5, or mixtures thereof.

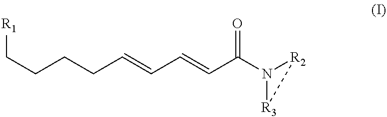

Preferably, the salivating agent comprises a material wherein R2 is 2-methyl-1-propyl and R3 is hydrogen, more preferably wherein R1 is Cl n-alkyl, R2 is 2-methyl-1-propyl and R3 is hydrogen. More preferably, the salivating agent comprises trans-pellitorin, a chemical having a structure according to formula (II):

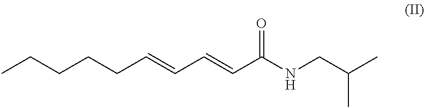

Some embodiments may comprise a TRPV1 activator, a transient receptor potential vanilloid receptor 1 activator. By adding a TRPV1 activator to an oral care composition with an off tasting component, the user of the composition may experience an improved taste over an oral care composition without the TRPV1 activator. Thus, the TRPV1 activator works to off-set the bad taste associated with many components used in oral care compositions. These activators may not only off-set bad tastes, but may also reduce dryness perception, by limiting the mouth's ability to perceive dryness. In one embodiment, the TRPV1 activator comprises vanillyl butyl ether, zingerone, capsaicin, capsiate, shoagol, gingerol, piperine, or a combination thereof. In one embodiment, a TRPV1 activator will be added in an amount of about 0.0001% to about 0.25% by weight of the oral care composition.

A sensate in the present invention may be or include a flavoring component. Suitable flavoring components include oil of wintergreen, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, cranberry, chocolate, green tea, and mixtures thereof.

Coolants may also be a sensate or part of the flavoring component. Coolants suitable for the present compositions include the paramenthan carboxyamide agents such as N-ethyl-p-menthan-3-carboxamide (known commercially as WS-3, WS-23, WS-5), MGA, TK-10, Physcool, and mixtures thereof. Other coolants may include those listed in US 2008/0008660, the entire substance of which is incorporated by reference herein. The listed cooling agents may also be used in combination with one another: 1-menthol, d-menthol, racemic menthol, menthone glycerol acetal (trade name: Frescolat®MGA), menthyl lactate (trade name: Frescolat®ML), wherein preferably menthyl lactate is 1-menthyl lactate, especially 1-menthyl-1-lactate), substituted menthyl-3-carboxylic acid amides (e.g. menthyl-3-carboxylic acid N-ethylamide), 2-isopropyl-N-2,3-trimethyl butanamide, substituted cyclohexanecarboxylic acid amides, 3-menthoxypropane-1,2-diol, 2-hydroxyethylmenthyl carbonate, 2-hydroxypropylmenthyl carbonate, N-acetylglycine menthyl ester, isopulegol, menthylhydroxycarboxylic acid esters (e.g. menthyl-3-hydroxybutyrate), monomenthyl succinate, 2-mercaptocyclodecanone, menthyl-2-pyrrolidin-5-one carboxylate, 2,3-dihydroxy-p-menthane, 3,3,5-trimethylcyclohexanone glycerol ketal, 3-menthyl-3,6-di- and trioxaalkanoates, 3-menthyl methoxyacetate, icilin.

On account of their particularly synergistic effect, other cooling agents may be: 1-menthol, d-menthol, racemic menthol, menthone glycerol acetal (trade name: Frescolat®MGA), menthyl lactate, preferably 1-menthyl lactate, in particular 1-menthyl-1-lactate (trade name: Frescolat®ML), substituted menthyl-3-carboxylic acid amides (e.g. menthyl-3-carboxylic acid N-ethyl amide), 2-isopropyl-N-2,3-trimethyl butanamide, substituted cyclohexanecarboxylic acid amides, 3-menthoxypropane-1,2-diol, 2-hydroxyethylmenthyl carbonate, 2-hydroxypropylmenthyl carbonate, isopulegol. Other cooling agents may be: 1-menthol, racemic menthol, menthone glycerol acetal (trade name: Frescolat®MGA), menthyl lactate (preferably 1-menthyl lactate, in particular 1-menthyl-1-lactate, trade name: Frescolat®ML), 3-menthoxypropane-1,2-diol, 2-hydroxyethylmenthyl carbonate, 2-hydroxypropylmenthyl carbonate. Other cooling agents may be: 1-menthol, menthone glycerol acetal (trade name: Frescolat®MGA), menthyl lactate (preferably 1-menthyl lactate, in particular 1-menthyl-1-lactate, trade name: Frescolat®ML).

Further nonlimiting examples of cooling agents may be found in WO2009/066262A1, including WS-23 (2-Isopropyl-N,2,3-trimethylbutyramide), WS-3 (N-Ethyl-p-menthane-3-carboxamide), WS-30 (1-glyceryl-p-menthane-3-carboxylate), WS-4 (ethyleneglycol-p-methane-3-carboxylate), WS-14 (N-t-butyl-p-menthane-3-carboxamide), WS-12 (N-(4-ethoxyphenyl)-p-menthane-3-carboxamide), WS-5 (Ethyl-3-(p-menthane-3-carboxamido)acetate, Menthone glycerol ketal (sold as Frescolat® MGA by Haarmann & Reimer), (−)-Menthyl lactate (sold as Frescolat® ML by Haarmann & Reimer), (−)-Menthoxypropane-1,2-diol (sold as Coolant Agent 10 by Takasago International), 3-(1-menthoxy)propane-1,2-diol, 3-(1-Menthoxy)-2-methylpropane-1,2-diol, (−)-Isopulegol is sold under the name “Coolact P®” by Takasago International., cis & trans p-Menthane-3,8-diols (PMD38)—Takasago International, Questice (menthyl pyrrolidone carboxylate), (1R,3R,4S)-3-menthyl-3,6-dioxaheptanoate—Firmenich, (1R,2S,5R)-3-menthyl methoxyacetate—Firmenich, (1R,2S,5R)-3-menthyl 3,6,9-trioxadecanoate—Firmenich, (1R,2S,5R)-menthyl 11-hydroxy-3,6,9-trioxaundecanoate—Firmenich, (1R,2S,5R)-3-menthyl (2-hydroxyethoxy)acetate—Firmenich, Cubebol—Firmenich, Icilin also known as AG-3-5, chemical name 1-[2-hydroxyphenyl]-4-[2-nitrophenyl-]-1,2,3,6-tetrahydropyrimidine-2-one), 4-methyl-3-(1-pyrrolidinyl)-2[5H]-furanone, Frescolat ML—menthyl lactate, Frescolat MGA—menthone glycerin acetal, Peppermint oil, Givaudan 180, L-Monomenthyl succinate, L-monomenthyl glutarate, 3-1-menthoxypropane-1,2-diol—(Coolact 10), 2-1-menthoxyethanol (Cooltact 5).

Sweetening agents can be added to the compositions as a sensate. These include saccharin, dextrose, sucrose, sucralose, lactose, xylitol, maltose, levulose, aspartame, sodium cyclamate, D-tryptophan, dihydrochalcones, acesulfame, sucralose, neotame, and mixtures thereof. Various coloring agents may also be incorporated in the present invention. Sweetening agents are generally used at levels of from about 0.005% to about 5%, by weight of the composition.

Sensates are generally used in the oral care compositions at levels of from about 0.001% to about 5%, by weight of the oral care composition. The sensate composition will preferably be present in an amount of from about 0.01% to about 4%, more preferably from about 0.1% to about 3%, and more preferably from about 0.5% to about 2% by weight.

### Antibacterial Agents and Anti-Inflammatory Agents

The present invention may comprise antibacterial and anti-inflammatory agents. One possible antibacterial agent may be a stannous ion source. The source of stannous ions may be a stannous salt. Stannous salts include stannous fluoride, stannous chloride dihydrate, stannous acetate, stannous gluconate, stannous oxalate, stannous sulfate, stannous lactate, stannous tartrate, or mixtures thereof. In certain embodiments, the stannous ion source comprises stannous fluoride. Stannous salts have been found to help in the reduction of caries, gingivitis, plaque, and sensitivity, and in providing breath benefits.

In some embodiments, the composition comprises the combined stannous ion source combined in an amount from about 0.1% to about 11%, by weight of the total composition. In other embodiments, the stannous ion source is present in an amount from about 0.5% to about 7%, in certain embodiments from about 1% to about 5%, and in some embodiments from about 1.5% to about 3% by weight of the total composition.

There are two key measurements of stannous that are relevant for the present invention. First is the stability of the stannous, as measured by the concentration of soluble stannous. The second is the bioavailability of the stannous, as measured using i-PGRM testing that determines a percent of antibacterial activity. Generally, the stannous must demonstrate a certain amount of stability, or concentration, before it is capable of being bioavailable. That is, for stannous, stability is a prerequisite for bioavailability. But higher stability of stannous does not necessarily mean higher bioavailability, indicating that maximizing the bioavailability of stannous requires careful balancing of ingredients.

Generally, in order to provide maximum therapeutic efficacy, the stannous must be bioavailable. In oral care, bioavailable stannous is achieved when stannous ions dissociate from the stannous salt and then proceed to chemically interact with the tooth surface and/or gum tissue. Given the relatively short time period when toothpaste is applied and brushed against the teeth and the relatively low general solubility and dissociation of stannous, an available amount of free stannous ions in a toothpaste formulation at the moment of application to the teeth is highly desirable.

In general, research has demonstrated that effective gingivitis efficacy can be anticipated for oral care compositions containing stannous ions that provide at least about 20% antibacterial activity, as determined using i-PGRM testing. In some embodiments, the antibacterial activity is at least about 30%, in some embodiments the antibacterial activity is at least about 40%, in some embodiments the antibacterial activity is at least about 50%, in some embodiments the antibacterial activity is at least about 60%, in other embodiments the antibacterial activity is at least about 65%, in another embodiment the antibacterial activity is at least about 70%, in some embodiments the antibacterial activity is at least about 75%, and in certain embodiments the antibacterial activity is at least about 80%, as determined using i-PGRM. In other embodiments, the antibacterial activity is at least about 85%, and in still other embodiments, it is at least about 90%, as determined using i-PGRM.

The following data in Table 4 shows the antibacterial activity of various dentifrices, as measured using the i-PGRM test method described herein. The negative control, which has 0.00% antibacterial activity, is commercially available Crest Cavity Protection, which contains no stannous and no SLS (sodium lauryl sulfate). The positive control, which has 100.00% antibacterial activity, is commercially available Crest Gum Care, which has a high level of stannous. Also tested were formulas A, B, and D from the examples given herein, all of which contain 0.1% by weight of the composition of PEO with a molecular weight of 4,000,000. Formula A further contains a high amount of stannous and SLS. Formula B contains a high amount of stannous and no SLS. Formula D contains a low amount of stannous and no SLS. In addition, the data shows the % antibacterial activity for two toothpastes that are commonly known and available and marketed as toothpastes to treat dry mouth.

TABLE 4

| | Final pH | Δ pH | SD (standard | % Antibacterial |
|---|---|---|---|---|
| | (mean) | (mean) | deviation) | Activity |
| Negative Control | 4.92 | 1.80 | 0.12 | 0.00 |
| (Crest Cavity | | | | |
| Protection) | | | | |
| Positive Control | 5.79 | 0.94 | 0.14 | 100.00 |
| Formula A | 5.63 | 1.21 | 0.17 | 93.3 |
| Formula B | 5.17 | 1.56 | 0.24 | 28.32 |
| Formula D | 5.17 | 1.55 | 0.42 | 28.90 |
| Commercial Dry | 4.59 | 2.13 | 0.06 | −37.86 |
| Mouth Toothpaste | | | | |
| #1 | | | | |
| Commercial Dry | 4.55 | 2.17 | 0.17 | −42.49 |
| Mouth Toothpaste | | | | |
| #2 | | | | |

The data in table 4 demonstrates that formulas containing high molecular PEO's have superior anti-bacterial activity compared to commercial dry mouth toothpastes that claim anti-bacterial benefits. In addition, the data shows that commercial toothpastes marketed as treating dry mouth have poor anti-bacterial activity. Given that part of effectively treating and lessening the effects of dry mouth is to free the mouth of harmful bacteria that can cause infection and/or caries, compositions of the present invention can be expected to effectively prevent or reduce the effects of dry mouth.

Similarly, the data in table 5 demonstrates that a rinse formula containing high molecular weight PEO's has superior anti-bacterial activity compared to a commercial dry mouth rinse claiming anti-bacterial benefits. The data was measured using the i-PGRM test method described herein. The negative control, which has 0.00% antibacterial activity, is commercially available Crest Cavity Protection toothpaste. The positive control, which has 100.00% antibacterial activity, is commercially available Crest Gum Care toothpaste. Also tested were rinse formula 1 from the examples herein containing 0.025% PEO, and a commercially available dry mouth rinse.

TABLE 5

| | Final pH | Δ pH | SD (standard | % Antibacterial |
|---|---|---|---|---|
| | (mean) | (mean) | deviation) | Activity |
| Negative Control | 5.29 | 1.55 | 0.19 | 0.00 |
| Positive Control | 6.23 | 0.62 | 0.04 | 100.00 |
| Rinse Example 1 | 5.91 | 0.94 | 0.10 | 65.96 |
| Commercial Dry | 5.34 | 1.51 | 0.19 | 5.32 |
| Mouth Rinse | | | | |

Some embodiments may comprise a zinc salt. For example, insoluble or sparingly soluble zinc compounds, such as zinc oxide or zinc carbonate, can be used as the zinc source. Preferred zinc sources however are soluble zinc sources such as zinc chloride or zinc sulphate. More preferred zinc sources are those where the zinc is already combined with a suitable chelating agent in the form of a salt or other complex, such as zinc citrate, zinc gluconate, zinc lactate and zinc glycinate. Especially preferred sources of zinc ions are zinc citrate, zinc gluconate, zinc lactate and mixtures thereof.

The present compositions may include a quaternary ammonium antimicrobial agent to provide bactericidal efficacy, i.e., effectiveness in killing, and/or altering metabolism of, and/or suppressing the growth of microorganisms which cause topically-treatable infections and diseases of the oral cavity, such as plaque, caries, gingivitis, and periodontal disease. The antimicrobial quaternary ammonium compounds used in the compositions of the present invention include those in which one or two of the substituents on the quaternary nitrogen has a carbon chain length (typically alkyl group) from about 8 to about 20, typically from about 10 to about 18 carbon atoms while the remaining substituents (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethoylstearyl ammonium chloride, cetylpyridinium chloride, quaternized 5-amino-1,3-bis(2-ethyl-hexyl)-5-methyl hexa hydropyrimidine, benzalkonium chloride, benzethonium chloride and methyl benzethonium chloride are exemplary of typical quaternary ammonium antibacterial agents. Other compounds are bis[4-(R-amino)-1-pyridinium]alkanes as disclosed in U.S. Pat. No. 4,206,215, Jun. 3, 1980 to Bailey. The pyridinium compounds are the preferred quaternary ammonium compounds, particularly preferred being cetylpyridinium, or tetradecylpyridinium halide salts (i.e., chloride, bromide, fluoride and iodide). Most preferred is cetylpyridinium chloride (CPC).

The quaternary ammonium antimicrobial agents are included in the present invention at levels of at least about 0.01%, at least about 0.030%, or at least about 0.035%, by weight of the composition. Typically, the level may be from about 0.045% to about 1.0% or from about 0.05% to about 0.10% by weight of the composition.

In some embodiments, the composition may comprise CPC wherein the composition delivers at least about 100 ppm bioavailable CPC, as measured by DRA. In some embodiments, the composition may comprise CPC wherein the composition delivers at least about 324 ppm bioavailable CPC, as measured by DRA. In other embodiments, the composition may deliver at least about 360 ppm bioavailable CPC, as measured by DRA. Bioavailability of CPC may be at least about 60%, in some embodiments, at least about 72%, as measured by DRA. In some embodiments, the bioavailability of CPC may be at least about 80%, and in other embodiments, it may be at least about 90%.

Some embodiments may comprise an anti-inflammatory agent or an antibacterial agent having an anti-inflammatory activity, i.e., against one or more of inflammatory factors produced by the body (host) in response to bacterial infection in the oral cavity, including matrix metalloproteinases (MMP's), cyclooxygenase (COX), interleukin 1 (IL-1), IL-1b converting enzyme (ICE), IL-1 Receptor Associated Kinase (IRAK), TGF-β1, inducible nitric oxide synthase (iNOS), hyaluronidase, cathepsins, and nuclear factor-kappa B (NF-κB) to provide enhanced efficacy against bacteria-mediated diseases of the oral cavity. Compositions with these agents are effective in inhibiting and/or killing pathogenic oral bacteria and in mediating host reaction to the presence of these pathogens in the oral cavity as well as to the toxins, endotoxins, inflammatory cytokines and mediators released by or prompted by these pathogens. A list of agents having anti-inflammatory activity includes citral, vitamin compounds such as riboflavin, riboflavin phosphate, folic acid, cyanocobalamin (vitamin B12), and menadione (vitamin K3); curcuminoids such as curcumin, demethoxycurcumin, bismethoxycurcumin and tetrahydrocurcumin; oils and extracts from spices and botanicals such as clove, cinnamon, cassia, ginger, basil, coriander, cilantro and allspice which contain active compounds including cinnamaldehyde, cinnamic acid, guaiacol and derivatives such as eugenol, isoeugenol, dihydroeugenol, vanillyl butyl ether, vanillin (4-formyl-guaiacol), 5-propenylguaethol, 4-ethyl-2-methoxyphenol, 4-allyl-2-methoxyphenol acetate, and 4-methyl guaiacol; oils or extracts of thyme, oregano and sage containing thymol, carvacrol and carvacryl ethyl ether; neem oil; flavonoids and flavones such as baicalein, baicalin, wogonoside, wogonin, and quercetin; phenolics from plant sources such as tea and cranberry including catechin, gallocatechin gallate, epicatechin (EC), epigallocatechin (EGC), epigallocatechin gallate (EGCG), epicatechin gallate (ECG), theaflavine, thearubigins, anthocyanidins/proanthocyanidins and anthocyanins (e.g., cyanidin, delphinidin, pelargonidin, peonidin, malvidin and petunidin); tannic acid; gallic acid; ellagic acid; ellagitannins; hexamidine; and berberine. Agents with anti-inflammatory activity may be present in an amount from about 0.001% to about 10%, by weight of the composition.

Other antibacterial and/or anti-microbial agents may include, but are not limited to: 5-chloro-2-(2,4-dichlorophenoxy)-phenol, commonly referred to as triclosan; 8-hydroxyquinoline and its salts; copper II compounds, including, but not limited to, copper(II) chloride, copper(II) sulfate, copper(II) acetate, copper(II) fluoride and copper(II) hydroxide; phthalic acid and its salts including, but not limited to those disclosed in U.S. Pat. No. 4,994,262, including magnesium monopotassium phthalate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; iodine; sulfonamides; bisbiguanides; phenolics; delmopinol, octapinol, and other piperidino derivatives; niacin preparations; zinc or stannous ion agents; nystatin; grapefruit extract; apple extract; thyme oil; thymol; antibiotics such as augmentin, amoxicillin, tetracycline, doxycycline, minocycline, metronidazole, neomycin, kanamycin, cetylpyridinium chloride, and clindamycin; analogs and salts of the above; methyl salicylate; hydrogen peroxide; metal salts of chlorite; and mixtures of all of the above. In another embodiment, the composition comprises phenolic antimicrobial compounds and mixtures thereof.

Other antimicrobial agents may be, but are not limited to, essential oils. Essential oils are volatile aromatic oils which may be synthetic or may be derived from plants by distillation, expression or extraction, and which usually carry the odor or flavor of the plant from which they are obtained. Useful essential oils may provide antiseptic activity. Some of these essential oils also act as flavoring agents. Useful essential oils include but are not limited to citra, thymol, menthol, methyl salicylate (wintergreen oil), eucalyptol, carvacrol, camphor, anethole, carvone, eugenol, isoeugenol, limonene, osimen, n-decyl alcohol, citronel, a-salpineol, methyl acetate, citronellyl acetate, methyl eugenol, cineol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, tropolone derivatives such as hinokitiol, avender oil, mustard oil, phenol, phenyl salicylate, pine oil, pine needle oil, sassafras oil, spike lavender oil, storax, thyme oil, tolu balsam, terpentine oil, clove oil, and combinations thereof. In one embodiment the essential oils are selected from thymol, methyl salicylate, eucalyptol, menthol and combinations thereof.

The oral compositions of the present invention may include an anti-plaque agent such as stannous salts, copper salts, strontium salts, magnesium salts, copolymers of carboxylated polymers such as Gantrez or a dimethicone copolyol. The dimethicone copolyol is selected from C12 to C20 alkyl dimethicone copolyols and mixtures thereof. In one embodiment the dimethicone copolyol is cetyl dimethicone copolyol marketed under the Trade Name Abil EM90. The dimethicone copolyol in one embodiment can be present in a level of from about 0.001% to about 25%, in another embodiment from about 0.01% to about 5%, and in another embodiment from about 0.1% to about 1.5% by weight of the oral composition.

The present compositions may not require a preservative. If present, preservatives such as benzoic acid, sodium benzoate, sorbic acid or parabens may be used.

Antibacterial, antimicrobial, or anti-inflammatory components may be present from about 0.001% to about 20% by weight of the oral composition. In another embodiment, they generally comprise from about 0.1% to about 5% by weight of the oral compositions of the present invention.

### Orally-Acceptable Carrier

The carrier for the components of the present compositions may be any orally-acceptable vehicle suitable for use in the oral cavity. The carrier may comprise suitable cosmetic and/or therapeutic actives. Such actives include any material that is generally considered safe for use in the oral cavity and that provides changes to the overall appearance and/or health of the oral cavity, including, but not limited to, anti-calculus agents, fluoride ion sources, stannous ion sources, whitening agents, anti-microbial, anti-malodor agents, anti-sensitivity agents, anti-erosion agents, anti-caries agents, anti-plaque agents, anti-inflammatory agents, nutrients, antioxidants, anti-viral agents, analgesic and anesthetic agents, H-2 antagonists, and mixture thereof. When present, the level of cosmetic and/or therapeutic active in the oral care composition is, in one embodiment from about 0.001% to about 90%, in another embodiment from about 0.01% to about 50%, and in another embodiment from about 0.1% to about 30%, by weight of the composition.

The following is a non-limiting list of actives that may be used in the present invention.

### Fluoride Ion

The present invention may comprise a safe and effective amount of a fluoride compound (e.g. water soluble). The fluoride ion may be present in an amount sufficient to give a fluoride ion concentration in the composition at 25° C., and/or in one embodiment can be used at levels of from about 0.0025% to about 5.0% by weight, in another embodiment from about 0.005% to about 2.0% by weight, to provide anticaries effectiveness. A wide variety of fluoride ion-yielding materials can be employed as sources of soluble fluoride in the present compositions. Examples of suitable fluoride ion-yielding materials are disclosed in U.S. Pat. Nos. 3,535,421, and 3,678,154. Representative fluoride ion sources include: stannous fluoride, sodium fluoride, potassium fluoride, amine fluoride, sodium monofluorophosphate, zinc fluoride, and many others. In one embodiment the composition comprises stannous fluoride or sodium fluoride, as well as mixtures thereof. Fluoride ion may also be considered an antibacterial active, as it has been shown to effectively reduce bacterial activity in the mouth.

### Anticalculus Agent

Compositions of the present invention may also comprise an anti-calculus agent, which in one embodiment may be present from about 0.05% to about 50%, by weight of the composition, in another embodiment is from about 0.05% to about 25%, and in another embodiment is from about 0.1% to about 15%. The anti-calculus agent may be selected from the group consisting of polyphosphates (including pyrophosphates) and salts thereof; polyamino propane sulfonic acid (AMPS) and salts thereof; polyolefin sulfonates and salts thereof; polyvinyl phosphates and salts thereof; polyolefin phosphates and salts thereof; diphosphonates and salts thereof; phosphonoalkane carboxylic acid and salts thereof; polyphosphonates and salts thereof; polyvinyl phosphonates and salts thereof; polyolefin phosphonates and salts thereof; polypeptides; and mixtures thereof; polycarboxylates and salts thereof; carboxy-substituted polymers; and mixtures thereof. In one embodiment, the polymeric polycarboxylates employed herein include those described in U.S. Pat. No. 5,032,386. An example of these polymers that is commercially available is Gantrez from International Speciality Products (ISP). In one embodiment, the salts are alkali metal or ammonium salts. Polyphosphates are generally employed as their wholly or partially neutralized water-soluble alkali metal salts such as potassium, sodium, ammonium salts, and mixtures thereof. The inorganic polyphosphate salts include alkali metal (e.g. sodium) tripolyphosphate, tetrapolyphosphate, dialkyl metal (e.g. disodium) diacid, trialkyl metal (e.g. trisodium) monoacid, potassium hydrogen phosphate, sodium hydrogen phosphate, and alkali metal (e.g. sodium) hexametaphosphate, and mixtures thereof. Polyphosphates larger than tetrapolyphosphate usually occur as amorphous glassy materials. In one embodiment the polyphosphates are those manufactured by FMC Corporation, which are commercially known as Sodaphos (n≈6), Hexaphos (n≈13), and Glass H (n≈21, sodium hexametaphosphate), and mixtures thereof. The pyrophosphate salts useful in the present invention include, alkali metal pyrophosphates, di-, tri-, and mono-potassium or sodium pyrophosphates, dialkali metal pyrophosphate salts, tetraalkali metal pyrophosphate salts, and mixtures thereof. In one embodiment the pyrophosphate salt is selected from the group consisting of trisodium pyrophosphate, disodium dihydrogen pyrophosphate (Na₂H₂P₂O₇), dipotassium pyrophosphate, tetrasodium pyrophosphate (Na₄P₂O₇), tetrapotassium pyrophosphate (K₄P₂O₇), and mixtures thereof. Polyolefin sulfonates include those wherein the olefin group contains 2 or more carbon atoms, and salts thereof. Polyolefin phosphonates include those wherein the olefin group contains 2 or more carbon atoms. Polyvinylphosphonates include polyvinylphosphonic acid. Diphosphonates and salts thereof include azocycloalkane-2,2-diphosphonic acids and salts thereof, ions of azocycloalkane-2,2-diphosphonic acids and salts thereof, azacyclohexane-2,2-diphosphonic acid, azacyclopentane-2,2-diphosphonic acid, N-methyl-azacyclopentane-2,3-diphosphonic acid, EHDP (ethane-1-hydroxy-1,1-diphosphonic acid), AHP (azacycloheptane-2,2-diphosphonic acid), ethane-1-amino-1,1-diphosphonate, dichloromethane-diphosphonate, etc. Phosphonoalkane carboxylic acid or their alkali metal salts include PPTA (phosphonopropane tricarboxylic acid), PBTA (phosphonobutane-1,2,4-tricarboxylic acid), each as acid or alkali metal salts. Polyolefin phosphates include those wherein the olefin group contains 2 or more carbon atoms. Polypeptides include polyaspartic and polyglutamic acids.

### Whitening Agent

A whitening agent may be included as an active in the present compositions. The actives suitable for whitening are selected from the group consisting of alkali metal and alkaline earth metal peroxides, metal chlorites, perborates inclusive of mono and tetrahydrates, perphoshates, percarbonates, peroxyacids, and persulfates, such as ammonium, potassium, sodium and lithium persulfates, and combinations thereof. Suitable peroxide compounds include hydrogen peroxide, urea peroxide, calcium peroxide, carbamide peroxide, magnesium peroxide, zinc peroxide, strontium peroxide and mixtures thereof. In one embodiment the peroxide compound is carbamide peroxide. It should be noted that peroxide compounds offer not only a whitening benefit, but may also be used in the present invention to offer antibacterial benefits. Suitable metal chlorites include calcium chlorite, barium chlorite, magnesium chlorite, lithium chlorite, sodium chlorite, and potassium chlorite. Additional whitening actives may be hypochlorite and chlorine dioxide. In one embodiment the chlorite is sodium chlorite. In another embodiment the percarbonate is sodium percarbonate. In one embodiment the persulfates are oxones. The level of these substances is dependent on the available oxygen or chlorine, respectively, that the molecule is capable of providing to bleach the stain. In one embodiment the whitening agents may be present at levels from about 0.01% to about 40%, in another embodiment from about 0.1% to about 20%, in another embodiment form about 0.5% to about 10%, and in another embodiment from about 4% to about 7%, by weight of the composition.

### Nutrients

Nutrients may improve the condition of the oral cavity and can be included in the compositions of the present invention. Nutrients include minerals, vitamins, oral nutritional supplements, enteral nutritional supplements, and mixtures thereof. Useful minerals include calcium, phosphorus, zinc, manganese, potassium and mixtures thereof. Vitamins can be included with minerals or used independently. Suitable vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Oral nutritional supplements include amino acids, lipotropics, fish oil, and mixtures thereof. Amino acids include, but are not limited to L-Tryptophan, L-Lysine, Methionine, Threonine, Levocarnitine or L-carnitine and mixtures thereof. Lipotropics include, but are not limited to, choline, inositol, betaine, linoleic acid, linolenic acid, and mixtures thereof. Fish oil contains large amounts of Omega-3 (N-3) polyunsaturated fatty acids, eicosapentaenoic acid and docosahexaenoic acid. Enteral nutritional supplements include, but are not limited to, protein products, glucose polymers, corn oil, safflower oil, medium chain triglycerides. Minerals, vitamins, oral nutritional supplements and enteral nutritional supplements are described in more detail in Drug Facts and Comparisons (loose leaf drug information service), Wolters Kluer Company, St. Louis, Mo., ©1997, pps. 3-17 and 54-57.

### Antioxidants

Antioxidants are generally recognized as useful in oral compositions. Antioxidants are disclosed in texts such as Cadenas and Packer, The Handbook of Antioxidants, © 1996 by Marcel Dekker, Inc. Antioxidants useful in the present invention include, but are not limited to, Vitamin E, ascorbic acid, Uric acid, carotenoids, Vitamin A, flavonoids and polyphenols, herbal antioxidants, melatonin, aminoindoles, lipoic acids and mixtures thereof.

### Analgesic and Anesthetic Agents

Anti-pain or desensitizing agents can also be present in the oral compositions of the present invention. Analgesics are agents that relieve pain by acting centrally to elevate pain threshold without disturbing consciousness or altering other sensory modalities. Such agents may include, but are not limited to: strontium chloride; potassium nitrate; sodium fluoride; sodium nitrate; acetanilide; phenacetin; acertophan; thiorphan; spiradoline; aspirin; codeine; thebaine; levorphenol; hydromorphone; oxymorphone; phenazocine; fentanyl; buprenorphine; butaphanol; nalbuphine; pentazocine; natural herbs, such as gall nut; Asarum; Cubebin; Galanga; scutellaria; Liangmianzhen; and Baizhi. Anesthetic agents, or topical analgesics, such as acetaminophen, sodium salicylate, trolamine salicylate, lidocaine and benzocaine may also be present. These analgesic actives are described in detail in Kirk-Othmer, Encyclopedia of Chemical Technology, Fourth Edition, Volume 2, Wiley-Interscience Publishers (1992), pp. 729-737.

### H-1 and H-2 Antagonists and Antiviral Actives

The present invention may also optionally comprise selective H-1 and H-2 antagonists including compounds disclosed in U.S. Pat. No. 5,294,433.

Antiviral actives useful in the present composition include any know actives that are routinely use to treat viral infections. Such anti-viral actives are disclosed in Drug Facts and Comparisons, Wolters Kluer Company, ©1997, pp. 402(a)-407(z). Specific examples include anti-viral actives disclosed in U.S. Pat. No. 5,747,070, issued May 5, 1998. Said Patent discloses the use of stannous salts to control viruses. Stannous salts and other anti-viral actives are described in detail in Kirk & Othmer, Encyclopedia of Chemical Technology, Third Edition, Volume 23, Wiley-Interscience Publishers (1982), pp. 42-71. The stannous salts that may be used in the present invention would include organic stannous carboxylates and inorganic stannous halides. While stannous fluoride may be used, it is typically used only in combination with another stannous halide or one or more stannous carboxylates or another therapeutic agent.

### Chelant

Chelating agents are able to complex calcium found in the cell walls of bacteria and can help to disrupt plaque by removing calcium from the calcium bridges which help hold this biomass intact. Suitable chelating agents include polycarboxylic acids and salts thereof such as tartaric acid, citric acid, gluconic acid, malic acid; polyphosphates and salts thereof, such as polyphosphates with n=2 or more; copolymers of carboxylic polymers such as Gantrez; copolymers of phosphate polymers and salts thereof, and alkyl phosphates. In another embodiment the composition comprises mixtures or combinations of chelating agents.

### Rinse Carrier

The carriers or excipients of rinse formulations of the present invention can include the usual and conventional components of mouthrinses and mouth sprays as disclosed in, e.g., U.S. Pat. No. 3,988,433 to Benedict (e.g., water, flavoring and sweetening agents, etc.). Carriers suitable for the preparation of compositions of the present invention are well known in the art. Their selection will depend on secondary considerations like taste, cost, and shelf stability, etc. Components of mouthrinses and mouth sprays typically include one or more of water (from about 60% to about 95%), ethanol (from about 0% to about 30%), a humectant (from about 5% to about 30%), a flavoring agent (from about 0.04% to about 2%), a sweetening agent (from about 0.01% to about 3%), and a coloring agent (from about 0.001% to about 0.5%). Such mouthrinses and mouth sprays may also include one or more of an anticaries agent (from about 0.05% to about 0.3% as fluoride ion) or an anticalculus agent (from about 0.1% to about 3%).

Another preferred composition of the subject invention is a dental solution. Components of dental solutions generally include one or more of water and humectant (from about 90% to about 99%), thickening agent (from 0% to about 5%), flavoring agent (from about 0.04% to about 2%), and sweetening agent (from about 0.1% to about 3%).

For rinses, a pharmaceutically-acceptable liquid carrier comprises a major proportion of water and humectant. The humectant serves to give compositions a moist feel to the mouth, and for particular humectants, to impart desirable sweetness of flavor. The humectant, on a pure humectant basis, may comprise from about 5% to about 30% or from about 7% to about 25%, by weight of the compositions in certain embodiments. Suitable humectants for use in compositions of the subject invention include edible polyhydric alcohols such as glycerin.

In some rinse embodiments, the composition may be substantially free of a humectant, such as glycerin, or may be less than about 6%, by weight, of the composition. In some embodiments, especially in ones with low levels of glycerin or substantially no glycerin, sucralose may be added to add sweetness that is reduced by the lower amount or lack of glycerin. In the low level or no glycerin embodiments, the high molecular weight PEO's contribute to a consumer-acceptable mouthfeel and aesthetics, along with the viscosity.

Water employed in the preparation of commercially suitable oral compositions should preferably be of low ion content and free of organic impurities. Water generally comprises from about 60% to about 95%, and typically from about 75% to about 93%, by weight of the composition herein. These amounts of water include the free water which is added plus that which is introduced with other materials, such as with the humectant.

For mouthrinse compositions the pH of the composition may range from about pH 3.0 to about pH 10.0. In a number of embodiments, the pH of the composition is from about 5.0 to about 8.0. The pH of the present compositions may be adjusted through the use of buffering agents. Buffering agents, as used herein, refer to agents that can be used to adjust the pH of the compositions to a range of about pH 3.0 to about pH 10.0. Buffering agents include monosodium phosphate, dibasic sodium phosphate, trisodium phosphate, sodium hydroxide, sodium carbonate, sodium acid pyrophosphate, citric acid, and sodium citrate. Buffering agents can be administered at a level of from about 0.5% to about 10%, by weight of the present compositions.

The present mouthrinse compositions are desirably clear for aesthetic reasons. By “clear” as used herein does not mean colorless, but means substantially lacking the presence of particles of sufficient size to scatter visible light as detected visually.

The composition may also be essentially free of ethyl alcohol and of organic solvents such as polypropylene glycol, butylene glycol and polyethylene glycol, which are normally employed as a carrier for water insoluble components such as flavoring oils.

### Additional Actives

Additional actives suitable for use in the present invention may include, but are not limited to, insulin, steroids, herbal and other plant derived remedies. Additionally, anti-gingivitis or gum care agents known in the art may also be included. Components which impart a clean feel to the teeth may optionally be included. These components may include, for example, baking soda or Glass-H. Also, it is recognized that in certain forms of therapy, combinations of these above-named agents may be useful in order to obtain an optimal effect. Thus, for example, an anti-microbial and an anti-inflammatory agent may be combined in a single composition to provide combined effectiveness.

Optional agents to be used include such known materials as synthetic anionic polymers, including polyacrylates and copolymers of maleic anhydride or acid and methyl vinyl ether (e.g., Gantrez), as described, for example, in U.S. Pat. No. 4,627,977, as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof. Additionally, the composition can include a polymer carrier, such as those described in U.S. Pat. Nos. 6,682,722 and 6,589,512 and U.S. application Ser. Nos. 10/424,640 and 10/430,617.

### Buffering Agents

The oral compositions may contain a buffering agent. Buffering agents, as used herein, refer to agents that can be used to adjust the pH of the oral compositions to a range of about pH 3.0 to about pH 10. The buffering agents include alkali metal hydroxides, ammonium hydroxide, organic ammonium compounds, carbonates, sesquicarbonates, borates, silicates, phosphates, imidazole, and mixtures thereof. Specific buffering agents include monosodium phosphate, trisodium phosphate, sodium benzoate, benzoic acid, sodium hydroxide, potassium hydroxide, alkali metal carbonate salts, sodium carbonate, imidazole, pyrophosphate salts, citric acid, and sodium citrate. Buffering agents are used at a level of from about 0.1% to about 30%, preferably from about 0.1% to about 10%, and more preferably from about 0.3% to about 3%, by weight of the oral compositions.

Coloring agents may also be added to the present composition. The coloring agent may be in the form of an aqueous solution, preferably 1% coloring agent in a solution of water. Pigments, pealing agents, filler powders, talc, mica, magnesium carbonate, calcium carbonate, bismuth oxychloride, zinc oxide, and other materials capable of creating a visual change to the oral compositions may also be used. Color solutions and other agents generally comprise from about 0.01% to about 5%, by weight of the composition. Titanium dioxide may also be added to the present composition. Titanium dioxide is a white powder which adds opacity to the compositions. Titanium dioxide generally comprises from about 0.25% to about 5%, by weight of the composition.

### Abrasive Polishing Materials

An abrasive polishing material may also be included in the oral compositions. The abrasive polishing material contemplated for use in the compositions of the present invention can be any material that does not excessively abrade dentin. Typical abrasive polishing materials include silicas including gels and precipitates; aluminas; phosphates including orthophosphates, polymetaphosphates, and pyrophosphates; and mixtures thereof. Specific examples include dicalcium orthophosphate dihydrate, calcium pyrophosphate, tricalcium phosphate, calcium polymetaphosphate, insoluble sodium polymetaphosphate, hydrated alumina, beta calcium pyrophosphate, calcium carbonate, and resinous abrasive materials such as particulate condensation products of urea and formaldehyde, and others such as disclosed by Cooley et al in U.S. Pat. No. 3,070,510, issued Dec. 25, 1962. Mixtures of abrasives may also be used. If the oral composition or particular phase comprises a polyphosphate having an average chain length of about 4 or more, calcium containing abrasives and alumina are not preferred abrasives. The most preferred abrasive is silica.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, “Zeodent”, particularly the silica carrying the designation “Zeodent 119.” The types of silica dental abrasives useful in the toothpastes of the present invention are described in more detail in Wason, U.S. Pat. No. 4,340,583, issued Jul. 29, 1982. Silica abrasives are also described in Rice, U.S. Pat. Nos. 5,589,160; 5,603,920; 5,651,958; 5,658,553; and 5,716,601. The abrasive in the oral compositions described herein is generally present at a level of from about 6% to about 70% by weight of the composition. Preferably, oral compositions contain from about 10% to about 50% of abrasive, by weight of the oral composition.

In certain embodiments, fused silica may be added as an abrasive in the oral care compositions. In some embodiments, the fused silica abrasive is present at a level of up to about 70% by weight of the composition. In other embodiments, the oral care composition contains from about 10% to about 50% of the fused silica, by weight of the oral composition. Without being bound by theory it is believed that fused silica is less reactive than precipitated silica, because it lacks surface hydroxyl groups. Consequently, the fused silica reacts less with the other components, thereby leading to improved efficacy of the composition.

### Thickening Agents

Additional thickening agents, such as polymeric thickeners, may be utilized. Suitable thickening agents are carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents can include polymeric polyether compounds, e.g., polyethylene or polypropylene oxide (M.W. 300 to 1,000,000), capped with alkyl or acyl groups containing 1 to about 18 carbon atoms.

A suitable class of thickening or gelling agents includes a class of homopolymers of acrylic acid crosslinked with an alkyl ether of pentaerythritol or an alkyl ether of sucrose, or carbomers. Carbomers are commercially available from B.F. Goodrich as the Carbopol® series. Particularly the carbopols include Carbopol 934, 940, 941, 956, and mixtures thereof.

Copolymers of lactide and glycolide monomers, the copolymer having the molecular weight in the range of from about 1,000 to about 120,000 (number average), are useful for delivery of actives into the periodontal pockets or around the periodontal pockets as a “subgingival gel carrier.” These polymers are described in U.S. Pat. Nos. 5,198,220; 5,242,910; and 4,443,430.

Thickening agents in an amount from about 0% to about 15%, or from about 0.01% to about 6%, in another embodiment from about 0.1% to about 5%, by weight of the total oral composition, can be used.

### Humectant

A humectant can help to keep the oral composition from hardening upon exposure to air and provide a moist feel in the mouth. A humectant or additional solvent may be added to the oral composition. Suitable humectants for the present invention include water, edible polyhydric alcohols such as glycerin, sorbitol, xylitol, butylene glycol, polyethylene glycol, propylene glycol, erythritol, maltitol, isomalt, lactitol, diglycerin, hydrogenated starch hydrolysate (HSH), and combinations thereof. Sorbitol, glycerin, water, and combinations thereof are preferred humectants. The humectant may be present in an amount of from about 0.1% to about 99%, from about 0.5% to about 95%, and from about 1% to about 90%.

### Surfactants

A surfactant may be added to the oral composition. Surfactants, also commonly referred to as sudsing agents, may aid in the cleaning or foaming of the oral composition. Suitable surfactants are those which are reasonably stable and foam throughout a wide pH range. The surfactant may be anionic, nonionic, amphoteric, zwitterionic, cationic, or mixtures thereof.

Examples of anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Examples of other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, issued May 25, 1976. In some embodiments, the oral composition may comprise an anionic surfactant at a level of from about 0.025% to about 9%, from about 0.05% to about 5% in some embodiments, and from about 0.1% to about 1% in other embodiments.

Another suitable surfactant is one selected from the group consisting of sarcosinate surfactants, isethionate surfactants and taurate surfactants. Preferred for use herein are alkali metal or ammonium salts of these surfactants, such as the sodium and potassium salts of the following: lauroyl sarcosinate, myristoyl sarcosinate, palmitoyl sarcosinate, stearoyl sarcosinate and oleoyl sarcosinate. The sarcosinate surfactant may be present in the compositions of the present invention from about 0.1% to about 2.5%, or from about 0.5% to about 2% by weight of the total composition.

Cationic surfactants useful in the present invention include derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride; etc. Preferred compounds are the quaternary ammonium fluorides described in U.S. Pat. No. 3,535,421, Oct. 20, 1970, to Briner et al., where said quaternary ammonium fluorides have detergent properties. Certain cationic surfactants can also act as germicides in the compositions disclosed herein. Cationic surfactants such as chlorhexidine, although suitable for use in the current invention, are not preferred due to their capacity to stain the oral cavity's hard tissues. Persons skilled in the art are aware of this possibility and should incorporate cationic surfactants only with this limitation in mind.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include the Pluronics (for example, poloxamers of the form PEO-PPO-PEO), polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Suitable betaine surfactants are disclosed in U.S. Pat. No. 5,180,577 to Polefka et al., issued Jan. 19, 1993. Typical alkyl dimethyl betaines include decyl betaine or 2-(N-decyl-N,N-dimethylammonio) acetate, coco betaine or 2-(N-coc-N,N-dimethyl ammonio)acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, lauramidopropyl betaine and the like. The betaines of choice are preferably the cocoamidopropyl betaine and, more preferably, the lauramidopropyl betaine.

In some embodiments, the present compositions may be essentially free of anionic, nonionic or amphoteric surfactants, which may have a negative effect on bioavailability of quaternary ammonium antimicrobials, such as CPC, and thus, their bactericidal efficacy. By “essentially free of anionic, nonionic or amphoteric surfactants” as used herein, means the composition may comprise only such an amount of surfactant, which will not substantially impair the activity of the quaternary ammonium antimicrobial agent. Generally this means the composition may contain less than about 0.1% total surfactant by weight of the composition. Preferably, in such embodiments, the composition may contain less than 0.05%, more preferably less than 0.01% and most preferably 0% of anionic surfactant or amphoteric surfactant. Preferably the composition may contain less than about 0.1%, more preferably less than 0.06% of nonionic surfactant.

Another reason to eliminate or reduce certain surfactants in some embodiments is that some surfactants may cause irritation in the mouth, a consequence of which is dryness. For example, anionic surfactant SLS is known to cause irritancy in the mouth. This irritancy may cause dryness, the opposite effect that is desired, so some embodiments of the present invention may be substantially free of SLS or have less than about 0.5% SLS, less than 0.1%, less than 0.05%, or less than 0.01%, by weight of the composition.

### Method of Use

The present invention also relates to methods for cleaning and polishing teeth. The method of use herein comprises contacting a subject's dental enamel surfaces and oral mucosa with the oral compositions according to the present invention. The method of treatment may be by brushing with a dentifrice or rinsing with a dentifrice slurry or mouthrinse. Other methods include contacting the topical oral gel, mouthspray, or other form with the subject's teeth and oral mucosa.

In a number of embodiments, mouthrinse and mouthspray compositions comprise cetylpyridinium chloride (CPC) as the active antimicrobial component at a concentration of at least about 0.035%, typically from about 0.045% to about 1.0% and from about 0.05% to about 0.10%, by weight of the composition, preferred. For the method of treating diseases or conditions of the oral cavity, including breath malodor (as well as long lasting breath protection), a safe and effective amount of the CPC composition is applied to the gingival/mucosal tissue and/or the teeth, for example, by rinsing with a mouthrinse for at least about 10 seconds or from at least about 20 seconds, preferably from about 30 seconds to about 60 seconds. The method involves expectoration of most of the composition following such contact. The frequency of such contact is may be from about once per week to about four times per day, typically from about thrice per week to about three times per day, and preferably from about once per day to about twice per day. The period of such treatment typically ranges from about one day to a lifetime. For particular oral care diseases or conditions the duration of treatment depends on the severity of the oral disease or condition being treated, the particular delivery form utilized and the patient's response to treatment. If delivery to the periodontal pockets is desirable, such as for the treatment of periodontal disease, a mouthrinse can be delivered to the periodontal pocket using a syringe or water injection device. These devices are known to those skilled in the art. Devices of this type include “Water Pik” by Teledyne Corporation. After irrigating, the subject can swish the rinse in the mouth to also cover the dorsal tongue and other gingival and mucosal surfaces. The present compositions may be used in conjunction with other oral care products such as a toothpaste, non-abrasive gel, toothgel, etc.

### Test Methods

The test method used to generate the data of table 1 is as follows: Tare the balance and record the weights of the filter papers. Using a pipette, 1 gram of each polymer solution is added to the center of a filter paper (drop-by-drop). Final weight is recorded and monitored over time as the samples dry on lab bench at the same room temperature conditions.

For table 2, the test method is as follows: For “no rinse”: 1×1 cm cotton swatches (triplicates) are placed into a weigh boat where each is labeled according to treatment type and number. Initial weights are recorded for each sample. 500 uL of polymer solution is applied to each cotton swatch sample then reweighed as wet weight. Samples are allowed to dry overnight at room temperature conditions. Sample weights are recorded in the following morning as “no rinse”. Samples are then placed on a tray in the incubator at 37° C., 3.3% CO2, and approximately 90% humidity (the metal tray at the bottom of the incubator has plenty of distilled water in the pan to ensure constant humidity). Opening and closing the incubator door frequently causes parameters to shift so minimizing this will ensure proper control over parameter. Samples are incubated for 8 hours then reweighed. The weights represent the moisture uptake ability for the treated samples. For “with rinse”: Following the weighing, samples are rinsed for 5 seconds in 500 g of vortexing water, and placed into respective weigh boat. Samples are dried overnight at room temperature conditions. Samples are weighed and placed in the incubator the following morning similar to above described procedure and weights are recorded as “with rinse”.

### Antibacterial Activity Test Methods

The stannous ion concentration and bioavailability required for the provision of therapeutic actions may differ for different clinical actions, for example, caries vs. gingivitis. However, a minimum antimicrobial activity level may be established. It is especially important to maintain efficacy in compositions wherein binding of stannous occurs, since stannous binding can easily lead to loss of antimicrobial activity. Herein, the minimum efficacy provided by the stannous ion source is defined in terms of effects in producing metabolic inhibition of dental plaque bacterial biofilms, which are responsible for numerous undesirable intraoral conditions. Efficacy is thus defined in terms of a noticeable and significant reduction in in situ plaque metabolism as measured using the in vitro Plaque Glycolysis and Regrowth Model (i-PGRM), developed in our laboratories.

The i-PGRM has been demonstrated to provide a correlation to bioavailability of stannous ions required to produce clinical antimicrobial, antigingivitis and antiplaque activity. The efficacy of stannous-containing compositions for gingivitis can be directly compared to a stannous-containing dentifrice formulation, such as described in U.S. Pat. No. 5,004,597 to Majeti, et al. or to a marketed dentifrice containing stannous fluoride, Crest Gum Care™.

The i-PGRM is a technique where plaque is grown from human saliva and treated with agents designed to produce various levels of antimicrobial activity. The purpose of this technique is to provide a simple and quick method for determining whether compounds have a direct effect on the metabolic pathways that plaque microorganisms utilize for the production of toxins, which adversely affect gingival health. In particular, the model focuses on the production of organic acids including lactic, acetic, propionic, and butyric. This method utilizes plaque grown on polished glass rods, which have been dipped in saliva overnight, dipped in soy broth and sucrose for 6 hours, and then dipped in saliva again overnight. The plaque mass grown on the glass rods is then treated for 1 minute with a 3:1 water to dentifrice slurry. The mass is then placed in a soy broth/sucrose solution for 6 hours and the pH of the incubation solution is measured at the end of the 6 hours. Thus, there are measures of pre-incubation pH and post incubation pH for both test formulations and controls. This testing is typically done with a number of replicates to minimize experimental variances, and a mean pH is calculated from the replicates. Due to strong reactivity with saccharolytic organisms, compositions containing high levels of bioavailable stannous produce significant inhibition of plaque acid generation in the i-PGRM assay. This enables formulation variations to be compared for stability and bioavailability of stannous with relative ease.

Stannous fluoride and/or other stannous salts are found in the oral compositions described herein in an effective amount to provide the desired level of antibacterial activity. The desired antibacterial activity is measured relative to non-stannous containing formulations (negative control) and to stannous-containing formulations (positive control) such as described in U.S. Pat. No. 5,004,597 to Majeti et al.

The antibacterial activity is calculated according to the formula:

$$Antibacterial Activity = 100 \% \times \frac{( \begin{matrix}Test Product Mean pH - \\ Non Stannous Control Mean pH\end{matrix} )}{( \begin{matrix}Stannous Control Mean pH - \\ Non Stannous Control Mean pH\end{matrix} )}$$

In evaluating bioavailability and biological activity of quaternary ammonium antimicrobials, such as CPC, in mouthwash formulations, an in vitro Disk Retention Assay (DRA) can be used to estimate drug bioavailability as well as an ex vivo Plaque Glycolysis and Regrowth Model (PGRM) to assess biological activity (S. J. Hunter-Rinderle, et al., “Evaluation of Cetylpyridinium Chloride-Containing Mouthwashes Using In Vitro Disk Retention and Ex Vivo Plaque Glycolysis Methods,” J. Clin. Den., 1997, 8:107-113). These assays are recommended for use in the proposed OTC monograph (Federal Register Vol. 68, No. 103 Part 356, “Oral Health Care Drug Products For Over-The-Counter Human Use; Antigingivitis/Antiplaque Drug Products; Establishment of a Monograph: Proposed Rules”).

Results of assays using cetylpyridinium chloride as the quaternary ammonium antimicrobial are detailed below.

Cetylpyridinum chloride (CPC) is a quaternary ammonium compound with an aliphatic chain (C=16) and is classified as a cationic surface-active agent (The United States Pharmacopeia-23, The National Formulary 18, p. 329, 1995). As such, it has both a positively charged hydrophilic region and a hydrophobic region. CPC has been shown to possess antimicrobial activity against a number of oral bacteria (R. N. Smith, et al., “Inhibition of Intergeneric Co-aggregation Among Oral Bacteria by Cetylpyridinium Chloride, Chlorhexidine Digluconate and Octenidine Dihydrochloride,” J. of Periodontal Research, 1991, 26: 422-429). The mechanism of action of CPC is dependent upon the ability of this positively charged molecule to interact with negatively charged anionic sites on the bacterial cell walls.

Under physiological conditions, bacterial cells carry a net negative charge. When bacteria are exposed to CPC, the positively charged hydrophilic group associated with the negatively charged groups on the bacterial surface allow the hydrophobic portion of CPC to interact with the cell membrane resulting in leakage of cellular components, disruption of bacterial metabolism, inhibition of cell growth, and cell death (A. A. Scheie, “Modes of Action of Currently Known Chemical Antiplaque Agents Other Than Chlorhexidine,” J. Dent. Res. 1989, 68:1606-1616; R. N. Smith, et al. “Inhibition of Intergeneric Co-aggregation Among Oral Bacteria by Cetylpyridinium Chloride, Chlorhexidine Digluconate and Octenidine Dihydrochloride,” J. Period. Res., 1991. 26:422-429; J. J. Merianos, “Quaternary Ammonium Antimicrobial Compounds,” in Disinfection, Sterilization and Preservation, 1991, edited by S. S. Block, 4^{th} edition, pp. 225-255).

Critical to CPC's antimicrobial activity is the availability of its positively charged hydrophilic region to facilitate attachment to bacterial and mucosal surfaces. As presented, attachment to bacterial surfaces is necessary to achieve cell lysis during CPC exposure while binding to oral mucosal surfaces helps to establish a CPC reservoir during and after treatment. Common excipients, in particular surfactants added in commercial oral care formulations, can significantly diminish or even completely neutralize the antimicrobial activity of CPC(S. Jenkins, et al., “The Magnitude and Duration of the Effects of Some Mouthrinse Products on Salivary Bacteria Counts, J. Clin. Periodontol. 1994, 21: 474-485; M. Pader, “Mouthwash Formulation,” in Oral Hygiene Products and Practice. Cosmetic Science and Technology Series, 1988, pp. 489-516). The degree to which CPC's activity is decreased is determined by the choice and concentration of excipients added to the CPC formulation.

In evaluating CPC bioavailability and biological activity in mouthwash formulations, the DRA and PGRM assays are used. It has been demonstrated that within the appropriate classes of antimicrobial agents including the present quaternary ammonium compounds, DRA and PGRM testing results broadly correlate with clinical outcomes from treatment with oral care formulations containing such actives, e.g., changes in plaque and gingivitis scores.

### DRA Performance Test Method

This method is designed as a performance assay to analyze mouthrinse formulations containing from about 0.03% to about 0.1% CPC to quantitatively determine the “free” (“unbound”) or “bioavailable” level of CPC needed for clinical efficacy. The DRA assay measures the amount of CPC “binding” to standardized cellulose filter disks during filtration of an undiluted mouthrinse sample. The “bioavailable” CPC binds to the hydroxyl groups on the cellulose fiber during filtration while CPC, which has been rendered “non-bioavailable” (or “bound”)” through interactions with mouthrinse components, simply passes through the filter paper, i.e., the positive charge on the compound is no longer available for binding to the negatively charged cellulose disks. In this way, the DRA test provides an estimate of the amount of CPC available for binding to bacteria and mucosal surfaces during use of the mouthrinse. DRA measurements of CPC availability have been positively correlated to the results of in vitro microbiological assays and in vivo germ kill tests. Historically, cellulose fibers have been used in other applications to similarly monitor biological activity of drug actives (“Dairy Products” in Official Methods of Analysis of the Association of Chemical Analytical Chemists. 13^{th} ed., 1980, Chapter 16:256).

“Bioavailable” CPC is the amount of CPC bound to or adsorbed to cellulose disks. This is determined by measuring the differences in CPC concentration in the mouthrinse before and after exposure to standardized cellulose disks. The method has been validated and shown to perform with acceptable accuracy, precision, and selectivity.

Mouthrinse formulations comprising from about 0.035% to about 0.1% CPC would pass the DRA test if assay results show the level of bioavailable CPC to be >324 ppm. For example, a formulation comprising 0.05% CPC at 72% bioavailability would provide 360 ppm CPC. Testing of products containing bioavailable levels of CPC of 324 ppm demonstrates positive clinical (antigingivitis, antiplaque) outcomes. Determination of CPC bioavailability in a finished product is important to product performance as it readily defines the amount (concentration) of active available for deposition at the site of action. Because the positively charged (cationic) hydrophilic region is critical to antimicrobial activity, any formulation component that diminishes the activity of this cationic group or that competes with the group may inactivate the product.

Desirably, a formulation containing 0.05% CPC would have at least about 65% bioavailability to deliver at least about 324 ppm bioavailable CPC. A formulation containing a lower level of CPC such as 0.04% would need to have at least about 81% bioavailability to deliver the minimum required level of bioavailable CPC for efficacy.

### Non-Limiting Examples

The oral compositions illustrated in the following examples illustrate specific embodiments of the oral compositions of the present invention, but are not intended to be limiting thereof.

Examples A-D are examples of dentifrice formulations. Other modifications can be undertaken by the skilled artisan without departing from the spirit and scope of this invention.

| | Formula A | |
|---|---|---|
| | (Wt %) | Formula B |
| Stannous Fluoride, USP | 0.454 | 0.454 |
| Glycerin, USP | 36.844 | 36.794 |
| Sodium Polyphosphate, FCC | 13 | 14 |
| Silica Zeodent 119 | 12.5 | 16.67 |
| Silica Zeodent 109 | 12.5 | 8.33 |
| Propylene Glycol, USP (PG) | 7 | 7 |
| Polyethylene Glycol 300 (PEG-6) | 7 | 7 |
| Sodium Lauryl Sulfate 28% soln | 3.4 | — |
| Cocamidopropyl Betaine 30% Solution | — | 2.5 |
| Zinc Lactate Dihydrate | 2.5 | 2.5 |
| Tribasic Sodium Phosphate, FCC | 1.1 | 1.1 |
| Flavor, Teaberry | 1 | 1 |
| Sodium Gluconate, USP | 0.652 | 0.652 |
| Carrageenan Mixture | 0.6 | 0.6 |
| Saccharin Sodium, USP | 0.5 | 0.35 |
| Sucralose, NF | — | 0.1 |
| Xanthan Gum, NF | 0.25 | 0.25 |
| FD&C Blue #1 Color (1% Solution) | 0.3 | 0.3 |
| Polyethylene specks, white | 0.3 | 0.3 |
| Poly(ethylene) oxide 4,000,000( MW) | 0.1 | 0.1 |
| Total | 100 | 100 |

| | Wt % |
|---|---|
| Formula C | |
| Sodium Fluoride | 0.243 |
| Sorbitol Solution | 32.565 |
| Water Purified, USP | 25.332 |
| Silica Zeodent 119 | 15 |
| Silica Zeodent 109 | 7 |
| Carbopol Slurry | 6 |
| Sodium Acid Pyrophosphate | 2.47 |
| Xanthan Gum | 0.3 |
| CMC | 1.05 |
| Sodium hydroxide solution 50% FCC | 1.74 |
| Saccharin | 0.45 |
| Cocamidopropyl Betaine 30% Solution | 2.5 |
| Poloxamer 407 | 3.5 |
| Flavor, Spearmint | 1.3 |
| Coolant | 0.1 |
| Polyethylene specks, white | 0.35 |
| Poly(ethylene) oxide 4,000,000(MW) | 0.1 |
| Total | 100 |
| Formula D | |
| Sodium Flouride, USP | 0.243 |
| Water Purified, USP | 31.858 |
| Sorbitol Solution | 38.067 |
| Titanium Dioxide for Dentrifice USP | 0.525 |
| Silica, Dental Type NF | 2.5 |
| Amorphous Silica Gel | 12.5 |
| Cocamidopropyl Betaine 30% Solution | 2.5 |
| Zinc Citrate Dihydrate | 0.533 |
| Stannous Chloride Dihydrate, FCC | 1.16 |
| Phytic Acid Aqueous Solution | 0.8 |
| Sodium hydroxide solution 50% FCC | 1.15 |
| Flavor, Spearmint | 1 |
| Sodium Gluconate, USP | 1.064 |
| Carrageenan Mixture | 0.7 |
| CMC | 1.3 |
| HEC | 0.5 |
| Saccharin Sodium, USP | 0.5 |
| Poly(ethylene) oxide 4,000,000(MW) | 0.1 |
| FD&C Blue #1 Color (1% Solution) | 0.3 |
| Polyethylene specks, white | 0.3 |
| Poloxamer 407 | 2.4 |
| Total | 100 |

Examples 1 and 2 are rinse formulations. Other modifications can be undertaken by the skilled artisan without departing from the spirit and scope of this invention.

| | | Example 1 - | Example 2 - |
|---|---|---|---|
| Ingredients | GCAS# | wt % | wt % |
| Purifed Water | 10032106 | QS | QS |
| Glycerin | 10045671 | 5.0 | 7.5 |
| Poloxamer 407, NF | 10048508 | 0.06 | 0.06 |
| Teaberry Flavor | 10046067 | 0.12 | 0.12 |
| CPC | 10045571 | 0.074 | 0.074 |
| Polyethylene Oxide Sentry | | 0.025 | 0.005 |
| Polyox WSR 301, NF | | | |
| Sucralose | 10032225 | 0.015 | 0.015 |
| Saccharin Sodium | 10047932 | 0.025 | 0.025 |
| Methyl paraben | 10000675 | 0.02 | 0.02 |
| Propyl paraben | 10000818 | 0.005 | 0.005 |
| FD&C Blue #1 | 10045748 | 0.0005 | 0.0005 |

The following consumer data in Table 6 demonstrates that formula D of the examples above, a toothpaste containing 0.1% of a PEO with a molecular weight of 4,000,000, along with high stannous and no SLS, offers superior performance regarding dry mouth when compared to a leading commercial dry mouth toothpaste. For each toothpaste, over 190 self-identified dry mouth sufferers used the toothpaste exclusively for one week, then rated the products on a scale of 0 to 100 in the categories detailed in table 6. The consumers used a 5 point scale, for which 0 equaled “poor”, 25 equaled “fair”, 50 equaled “good”, 75 equaled “very good”, and 100 equaled “excellent”.

TABLE 6

| | Total Rep | |
|---|---|---|
| | Leading Commercial | |
| | Dry Mouth | |
| | Toothpaste | Formula D |
| Number of Consumers | 194 | 192 |
| Overall Rating | 45 | 69 |
| Favorable Comments | 27 | 48 |
| (Mouth + Breath Refreshment) | | |
| Unfavorable Comment | 21 | 11 |
| (Mouth + Breath Refreshment) | | |
| Unfavorable Comment | 26 | 7 |
| (Texture/Consistency) | | |
| Unfavorable Comment | 26 | 11 |
| (Foaming) | | |
| Better than Expected | 30 | 54 |
| Mouth Refreshment | 27 | 46 |
| Not as good as I expected | 52 | 16 |
| Unfav. Texture/Consistency | 25 | 12 |
| Cleans my Teeth Thoroughly | 57 | 76 |
| Cleans my Whole Mouth | 52 | 74 |
| Makes my Mouth Feel Moist | 49 | 71 |
| Long Lasting Dry Mouth Relief | 44 | 60 |
| Helps relieve my Dry Mouth | 47 | 62 |

The consumer data in table 7 below demonstrates the superiority of rinse example 1 formula above, comprising 0.025% PEO, when compared to the leading commercially available rinse marketed for dry mouth. The data was collected from 19 consumers after their use of the products.

TABLE 7

| | Rinse | Commercially Available |
|---|---|---|
| | example 1 | Dry Mouth Rinse |
| Overall Acceptance Rating (0-100) | 68 | 52 |
| Paste (0-10) | 6.8 | 5 |
| Teeth Feel (0-10) | 7 | 5.3 |
| whole mouth feel (0-10) | 7.3 | 6.1 |
| Mouth Moisture (0-10) | 7.1 | 6.1 |
| Foam (0-10) | 6.7 | 5 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it will be obvious to those skilled in the art that various changes and modifications may be made without departing from the spirit and scope of the invention. It is therefore intended to cover in the appended claims all such changes and modifications that are within the scope of the invention.

We claim:

1. An oral care composition for the treatment of dry mouth comprising:
(a) a polyethylene oxide with a molecular weight from about 200,000 to about 7,000,000;
(b) an antibacterial agent; and
(c) a sensate.

2. The oral care composition of claim 1, wherein the oral care composition is a rinse.

3. The oral care composition of claim 1, wherein the antibacterial agent comprises from about 0.035% to about 1% of cetylpyridinium chloride, by weight of the composition.

4. The oral care composition of claim 3, wherein the composition delivers at least about 360 ppm bioavailable cetylpyridinium chloride.

5. The oral care composition of claim 3, wherein the antibacterial agent further comprises peroxide.

6. The oral care composition of claim 1, wherein the sensate comprises a sweetener and/or a flavoring.

7. The oral care composition of claim 1, wherein the polyethylene oxide has a molecular weight from about 3,000,000 to about 5,000,000.

8. The oral care composition of claim 1, wherein the polyethylene oxide is from about 0.003% to about 0.05%, by weight of the composition.

9. The oral care composition of claim 1, wherein the polyethylene oxide is from about 0.005% to about 0.025%, by weight of the composition.

10. The oral care composition of claim 1, further comprising glycerin at a level of less than about 6%, by weight of the composition.

11. The oral composition of claim 10, further comprising sucralose.

12. The oral care composition of claim 1, wherein the oral care composition is substantially free of alcohol.

13. The oral care composition of claim 1, further comprising an anti-inflammatory agent.

14. A rinse for the treatment of dry mouth comprising:
(a) polyethylene oxide with a molecular weight from about 3,000,000 to about 5,000,000;
(b) an antibacterial agent comprising cetylpyridinium chloride, wherein the composition delivers at least about 360 ppm cetylpyridinium chloride;
(c) an anti-inflammatory agent; and
(d) a sensate comprising a sweetener and/or flavoring.

15. A method of treating dry mouth by administering to a subject's oral cavity a composition comprising:
(a) a polyethylene oxide with a molecular weight from about 200,000 to about 7,000,000;
(b) an antibacterial agent; and
(c) a sensate.
